(19) United States
(12) Patent Application Publication
(10) Pub. No.: US 2008/0127707 A1
(43) Pub. Date: Jun. 5, 2008

(54) Stent crimping assembly and method

(75) Inventors: Arkady Kokish, Santa Clara, CA (US); Daniel H. Shumer, Santa Clara, CA (US)

Correspondence Address: BEYER WEAVER LLP/Abbott Vascular Devices, P.O. BOX 70250, OAKLAND, CA, 94612-0250, US

(73) Assignee: Abbott Laboratories, Redwood City, CA (US)

(21) Appl. No.: 11/986,419

(22) Filed: Nov. 20, 2007

Related U.S. Application Data
(60) Provisional application No. 60/872,134, filed on Nov. 30, 2006.

(51) Int. Cl. B21D 41/04; A61F 2/06

(52) U.S. Cl. 72/402

(57) ABSTRACT

A stent crimping assembly is provided for crimping a stent from a first diameter to a reduced second diameter. The assembly includes a set of two or more blade devices each having a proximal portion, a downstream distal portion, and a respective edge wall extending from the proximal portion to the distal portion. Each respective edge wall is oriented relative to one another, in a respective crimp position, to collectively define an elongated conical-shaped crimp aperture. The conical-shaped crimp aperture at the respective proximal portion of each blade is formed for receipt of at least a portion of the stent in the first diameter. The crimping assembly further includes a drive assembly associated with each blade device and configured to independently displace each blade edge wall in a manner substantially along a respective predetermined first path from the respective crimp position to a respective retracted position, oriented a predetermined incremental distance from the crimp position in a respective proximal direction.

### RELATED APPLICATION DATA

The present application claims priority under 35 U.S.C. §119 to U.S. Provisional Application Ser. No. 60/872,134, naming Arkady Kokish et al as inventors, filed Nov. 30, 2006, and entitled STENT CRIMPING ASSEMBLY AND METHOD, which is incorporated herein by reference in its entirety and for all purposes.

### FIELD OF THE INVENTION

The present invention relates generally to intraluminal devices, and more particularly relates to apparatus and methods for diametrically reducing the size of these devices, such as a stent, stent-graft, graft or vena cava filter, for percutaneous transluminal delivery thereof.

### BACKGROUND OF THE INVENTION

A number of vascular diagnostic and interventional medical procedures are now performed translumenally. For example, a catheter is introduced into the vascular system at a convenient access location and guided through the vascular system to a target location using established techniques. Such procedures require vascular access, which is usually established during the well-known Seldinger technique. Vascular access is generally provided through an introducer sheath that is positioned to extend from outside the patient body, through a puncture in the femoral artery for example, and into the vascular lumen. Catheters or other medical devices are advanced into the patient's vasculature through the introducer sheath, and procedures such as balloon angioplasty, stent placement, etc. are performed.

In particular, stents and stent delivery assemblies are utilized in a number of medical procedures and situations, and as such their structure and function are well known. A stent is a generally cylindrical prosthesis introduced, via a catheter, into a lumen of a body vessel in a configuration having a generally reduced diameter for transport and delivery, and then expanded to a diameter of the target vessel when deployed. In its expanded configuration, the stent supports and reinforces the vessel walls while maintaining the vessel in an open, unobstructed condition.

Balloon expandable stents are well known and widely available in a variety of designs and configurations. Balloon expandable stents are crimped to their reduced diameter about the delivery catheter, then maneuvered to the deployment site and expanded to the vessel diameter by fluid inflation of a balloon positioned between the stent and the delivery catheter. One example of a stent is described in US Patent Application having Publication No. 2004/0093073, published May 13, 2004, the content of which is incorporated herein by reference.

During advancement of the stent through a body vessel to a deployment site, the crimped stent must capable of securely maintaining its axial position on the delivery catheter. That is, the crimped stent must not translocate proximally or distally during advancement, and especially must not dislodge from the catheter. Stents that are not properly crimped, secured or retained to the delivery catheter may slip and will either be lost, be deployed in the wrong location or only be partially deployed. Moreover, the stent must be crimped in such a way as to minimize or prevent distortion of the stent, and thereby, minimize or prevent abrasion and/or trauma to the vessel walls. Additionally, if a stent has been coated with a beneficial agent, care must be taken when crimping the stent onto the delivery device so that the coating is not disturbed or removed from the stent during the crimping process.

In the past, crimping has been performed by hand, often resulting in an undesirable application of uneven radial crimping forces to the stent. Such a stent must either be discarded or re-crimped. Stents that have been crimped multiple times can suffer from fatigue and may be scored or otherwise marked, increasing the risk of thrombosis. In fact, a poorly crimped stent can also damage the underlying balloon.

In addition to hand crimping of stents, automated crimping machines have been developed, wherein the automated crimping machines provide a more consistent crimp radial force during the crimping process or consistent profile. In addition to providing consistent crimping forces, many other crimping parameters can be closely controlled through the use of computer controls or mechanical controls. Typically, an automated crimping machine and related crimping methods include a crimp head comprising a plurality of segments aligned to defined a cylindrical-shaped cavity that is reduced in diameter in an iris-type displacement, uniformly compressing the stent. Typical of such designs are described in U.S. Pat. No. 6,629,350 to Motsenbocker.

While these designs are suitable to uniformly the crimp stents, the iris-type movement generally involves a rolling motion where the blades slide tangentially across an exterior or outer surface of the stent. Such sliding contact with the outer surface of some stents may be problematic, such as for example drug coated stents. Accordingly, it is desirable to provide a crimping apparatus that minimizes damage to the outer surface of the stent during the crimping process.

### SUMMARY OF THE INVENTION

The present invention provides an apparatus and methods for mechanically crimping a generally tubular stent from a first diameter to a reduced second diameter. A stent crimping assembly is provided that includes a set of two or more blade devices each having a proximal portion, a downstream distal portion, and a respective edge wall extending from the proximal portion to the distal portion. Each respective edge wall is oriented relative to one another, in a respective crimp position, to collectively define an elongated conical-shaped crimp aperture. Further, each edge wall tapers inwardly along a common longitudinal axis from the respective blade proximal portion toward the distal portion. The conical-shaped crimp aperture at the respective proximal portion of each blade is formed for receipt of at least a portion of the stent in the first diameter. The crimping assembly further includes a drive assembly associated with each blade device and configured to independently displace each blade edge wall in a manner substantially along a respective predetermined first path from the respective crimp position to a respective retracted position, oriented a predetermined incremental distance from the crimp position in a respective proximal direction.

In one specific embodiment, a transverse cross-sectional dimension of each respective edge wall is circular sector-shaped such that the collective transverse cross-sectional dimension of the opposed two or more blades, in the crimp position, is substantially circular shaped. Each respective edge walls, in the crimp position, is configured to cooperate with an adjacent edge wall to form a substantially continuous circular surface to define a substantially continuous crimp aperture.

After each blade device has sequentially displaced from the respective crimp position to the respective retracted position, the drive assembly is configured to displace the respective edge walls along respective predetermined second paths, as a unit, from the respective retracted position back to the crimp position. This movement displaces the stent substantially along the common longitudinal axis, relative to the crimping assembly, in a direction toward the respective distal portions.

In yet another arrangement, each respective first and second path of each respective edge wall is contained in a respective plane extending through the common longitudinal axis.

The respective predetermined first path each of each blade edge wall from the respective crimp position to the retracted position is substantially linear, in one embodiment, while each respective predetermined first path is tapered radially outwardly away the common longitudinal axis. In yet another configuration, the respective predetermined first path each of each blade edge wall from the respective crimp position to the retracted position is substantially non-linear, initially extending in a direction radially away from the common longitudinal axis.

In another aspect of the present invention, a stent crimping assembly is provided for crimping a stent from a first diameter to a reduced second diameter. The crimping assembly includes a set of two or more blade devices each having a first end, a second end, and a respective edge wall in opposed relationship to one another. Each respective edge wall tapers inwardly from the respective one ends to the respective second ends, relative to a common longitudinal axis of an elongated crimp aperture collectively defined by the opposed edge walls in a respective crimp position. Each respective one ends collectively defining a receiving port into the crimp aperture formed for receipt of at least a portion of the stent in the first diameter. A respective drive assembly, associated with each blade device, is configured to sequentially displace each blade substantially along a respective predetermined path from the respective crimp position to a respective retracted position. At this location, the blade device is oriented a predetermined incremental amount substantially along the common longitudinal axis of the crimp aperture and in a relative direction toward the respective one ends, sequentially diametrically reducing at least a portion of the stent from the first diameter to the reduced second diameter.

In one specific arrangement, after each blade device has sequentially displaced from the respective crimp position to the respective retracted position, each respective drive assembly is configured displace the respective blade devices substantially along the common longitudinal axis, as a unit, from the respective retracted position back to the crimp position. This unitary embodiment, the stent is displaced, relative to the crimping assembly, in the direction toward the respective second ends. Each respective drive assembly is configured to displace each respective blade device, from the respective crimp position to the respective retracted position, along the predetermined path in a direction radially away from the common longitudinal axis.

### BRIEF DESCRIPTION OF THE DRAWINGS

The assembly of the present invention has other objects and features of advantage which will be more readily apparent from the following description of the best mode of carrying out the invention and the appended claims, when taken in conjunction with the accompanying drawing, in which:

FIG. 1 is a front perspective view of a stent crimping assembly constructed in accordance with the present invention, and illustrating the blade devices thereof in a crimped condition.

FIG. 2 is a rear perspective view of the crimping assembly of FIG. 1.

FIG. 3 is an enlarged, fragmentary, front perspective view the crimping assembly of FIG. 1, illustrating a crimp aperture formed between the blade devices thereof, one blade device of which has been removed.

FIG. 4 is an enlarged side elevation of a blade main assembly of the crimping assembly of FIG. 1.

FIG. 5 is a bottom perspective view of the blade main assembly of FIG. 4.

FIG. 6 is an exploded bottom perspective view of the blade main assembly of FIG. 4.

FIG. 7 is an enlarged, fragmentary, rear perspective view of the crimping assembly of FIG. 1, illustrating an entrance port into the crimp aperture formed between the blade devices, in a crimp position.

FIG. 8A is an enlarged, fragmentary, front perspective view of the crimping assembly of FIG. 1, illustrating an exit port from the crimp aperture and the blade devices, in a crimp position.

FIG. 8B is a fragmentary, front perspective view of the crimping assembly of FIG. 8A, illustrating one blade device moved to a retracted position.

FIG. 8C is a fragmentary, front perspective view of the crimping assembly of FIG. 8B with the one moved blade device removed to illustrate stent/delivery device disposed in the crimp aperture for crimping thereof.

FIG. 8D is a fragmentary, front perspective view of the crimping assembly of FIG. 8C, illustrating a second blade device moved to a respective retracted position.

FIG. 8E is a fragmentary, front perspective view of the crimping assembly of FIG. 8C, illustrating a third blade device moved to a respective retracted position invention.

FIG. 8F is a fragmentary, front perspective view of the crimping assembly of FIG. 8C, illustrating a fourth blade device moved to a respective retracted position invention.

FIG. 8G is a fragmentary, front perspective view of the crimping assembly of FIG. 8A, illustrating all the blade devices in their respective retracted position.

FIG. 9A is an enlarged front elevation of the blade devices of the crimping assembly of FIG. 1, in a crimping position.

FIG. 9B is a front elevation of the blade devices of FIG. 9A, in a retracted position.

FIG. 10 is an enlarged, fragmentary, bottom plan view, in cross-section, of the blade device, taken along plane of the line 10-10 in FIG. 9A, and illustrating a substantially linear movement of the blade device along a first path and a second path.

FIG. 11 is a fragmentary, bottom plan view, in cross-section, of the vice of FIG. 10, illustrating an alternative embodiment of the first path.

### DETAILED DESCRIPTION

While the present invention will be described with reference to a few specific embodiments, the description is illustrative of the invention and is not to be construed as limiting the invention. Various modifications to the present invention can be made to the preferred embodiments by those skilled in the art without departing from the true spirit and scope of the invention as defined by the appended claims. It will be noted here that for a better understanding, like components are designated by like reference numerals throughout the various figures.

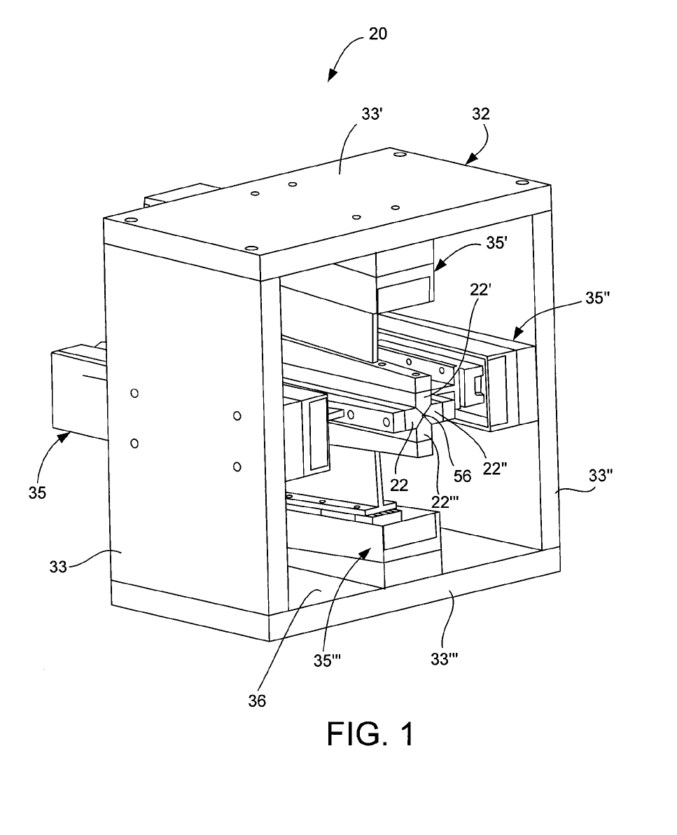

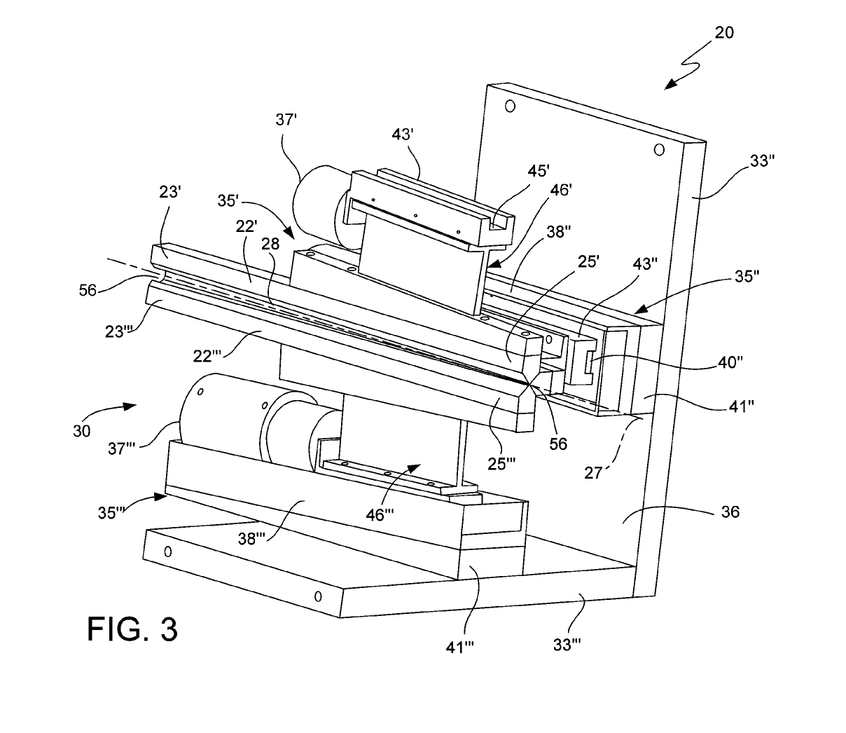

Referring now to FIGS. 1-4 and 7-8G, a stent crimping assembly, generally designated 20, is provided for crimping a stent 21 from a first diameter to a reduced second diameter. The crimping assembly 20 includes a set of two or more blade devices 22, 22′, 22″, . . . 22ⁿ (were n=total number of the blade devices) each having a respective proximal portion 23, 23′, 23″, . . . 23ⁿ, a respective distal portion 25, 25′, 25″, . . . 25ⁿ and a respective edge wall 26, 26′, 26″, . . . 26ⁿ extending from the proximal portion to the distal portion. Each respective edge wall is oriented radially about a common longitudinal axis 27 in a side-by-side relationship to one another, in a respective crimp position (FIGS. 1, 8A and 9A). Collectively, the adjacent edge walls define an elongated crimp aperture 28, each respective edge wall 26, 26′, 26″, . . . 26ⁿ of which tapers radially inward toward the common longitudinal axis 27 from the respective blade proximal portion 23, 23′, 23″, . . . 23ⁿ toward the distal portion 25, 25′, 25″, . . . 25ⁿ. This preferably conical frustum-shaped crimp aperture 28, at the respective proximal portion of each blade, is diametrically wider than that of the respective distal portion, and formed for receipt of at least a portion of the stent 21 in the first diameter.

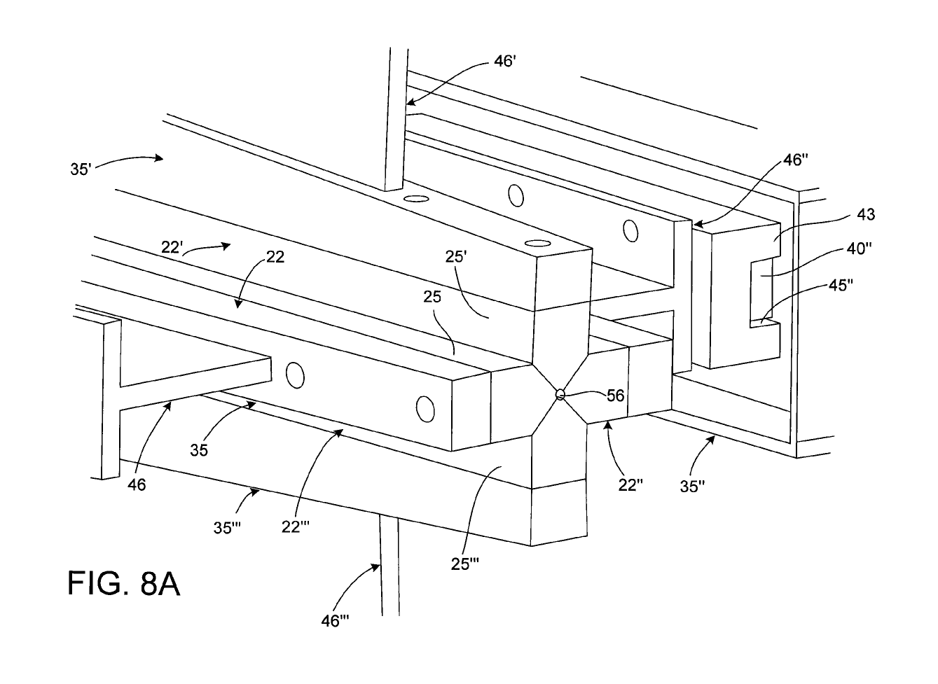

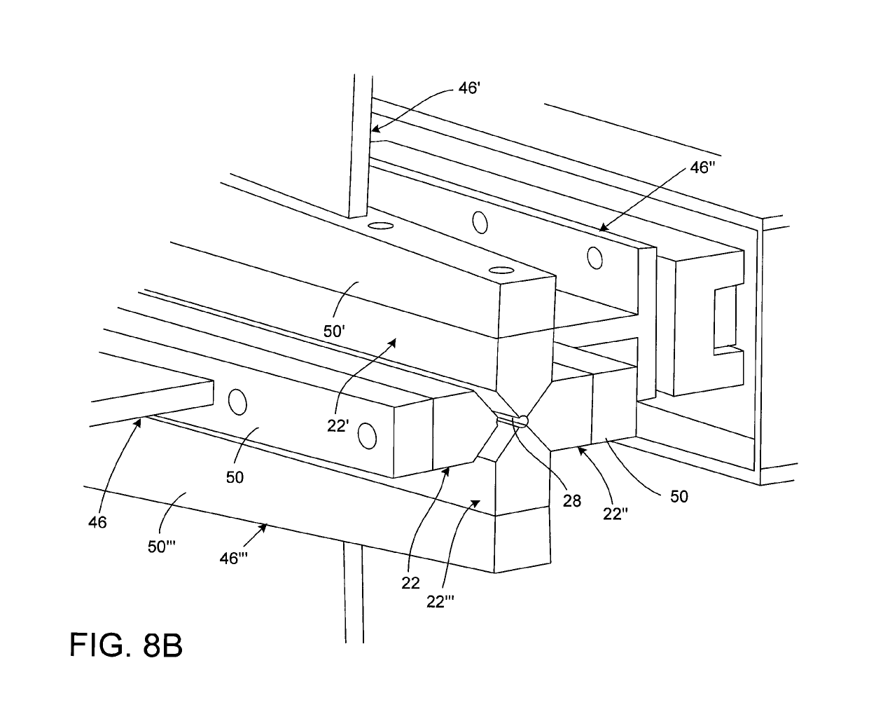

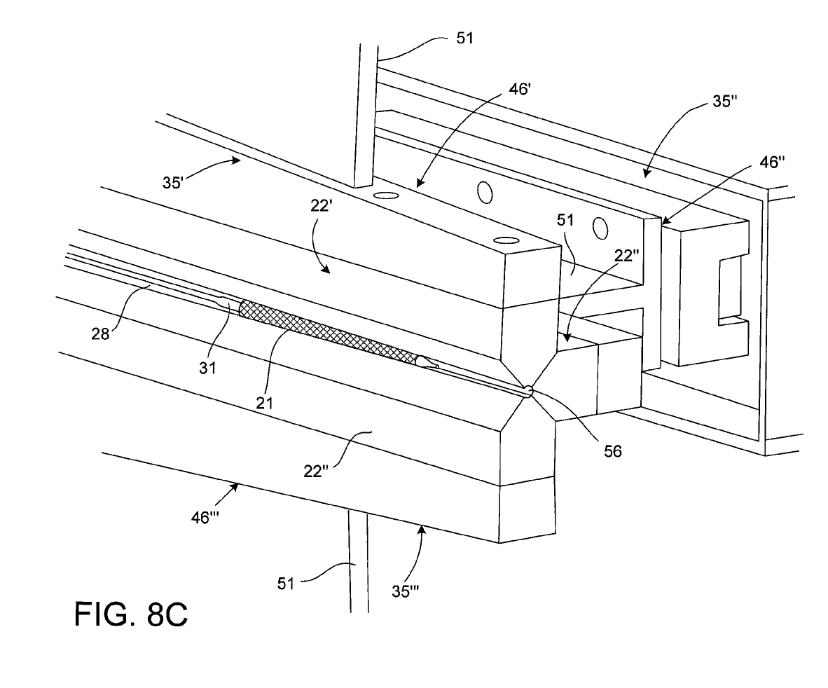

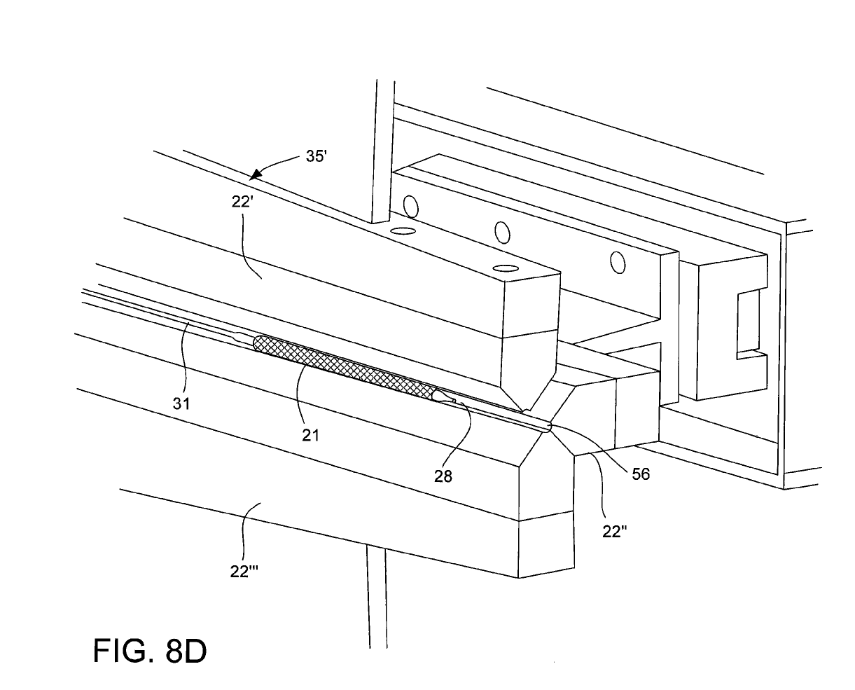

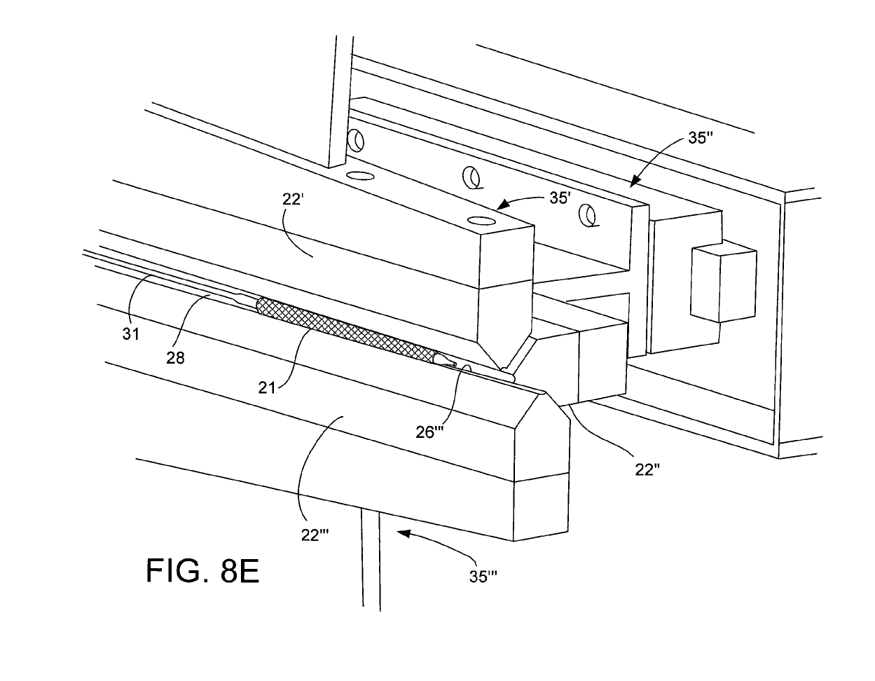

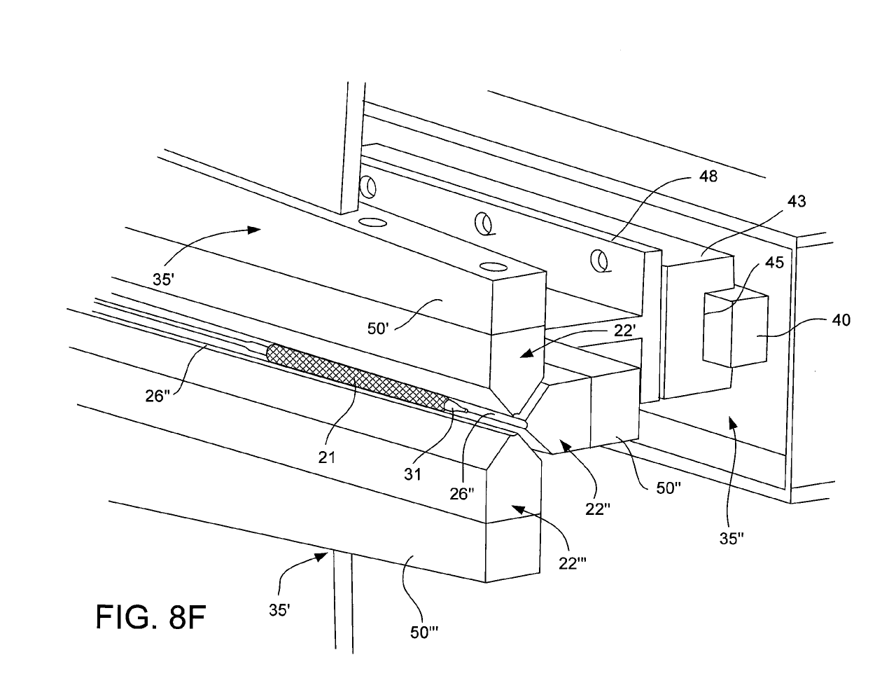

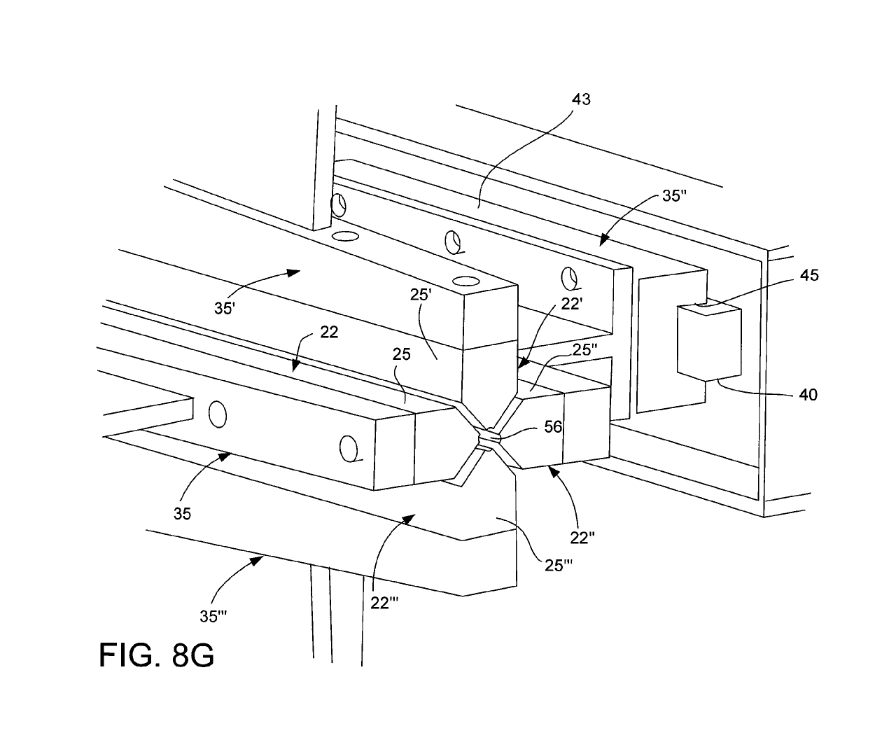

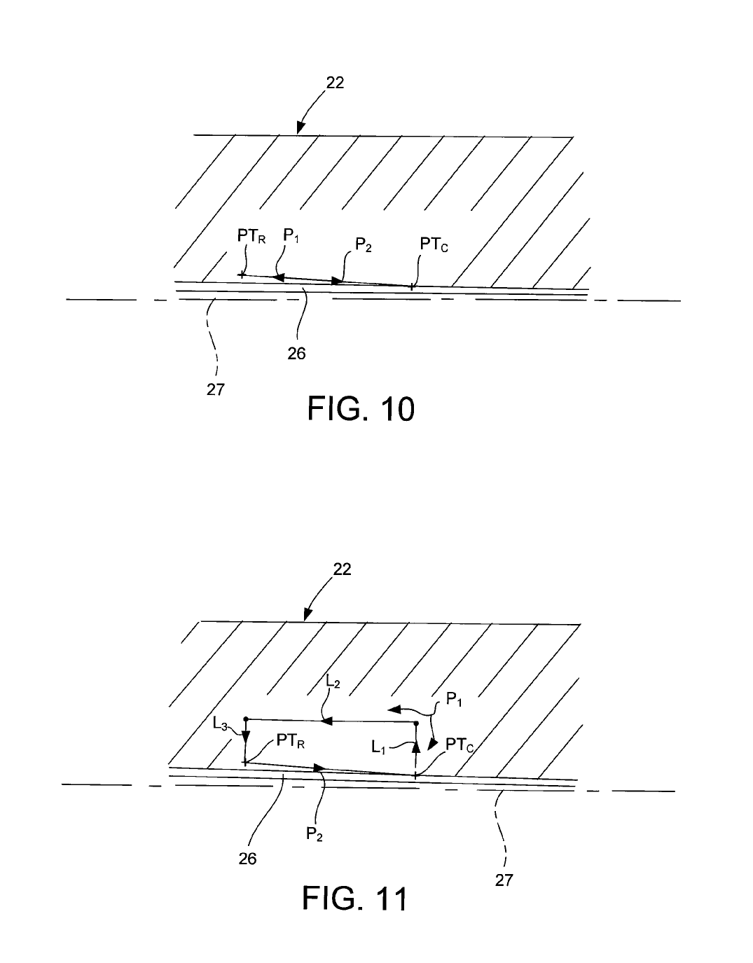

A drive assembly 30, associated with each blade device 22, 22′, 22″, . . . 22ⁿ, is configured to independently displace each respective blade edge wall 26, 26′, 26″, . . . 26ⁿ in a manner substantially along a respective predetermined first path P₁ (e.g., FIGS. 10, 11) from the respective crimp position (e.g., blade device 22 in FIG. 8A) to a respective retracted position (e.g., blade device 22 in FIG. 8B). The respective first path P₁ of each blade device 22, 22′, 22″, . . . 22ⁿ is contained in a respective plane extending through the longitudinal axis of the respective blade device and through a common longitudinal axis 27 of the crimp aperture 28. In the retracted position, each respective edge wall 26, 26′, 26″, . . . 26ⁿ is proximally spaced, generally longitudinally, from the crimping position, in same respective plane.

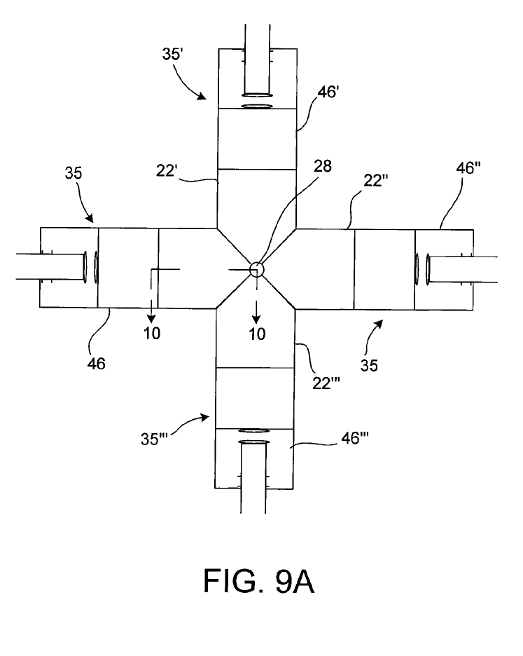

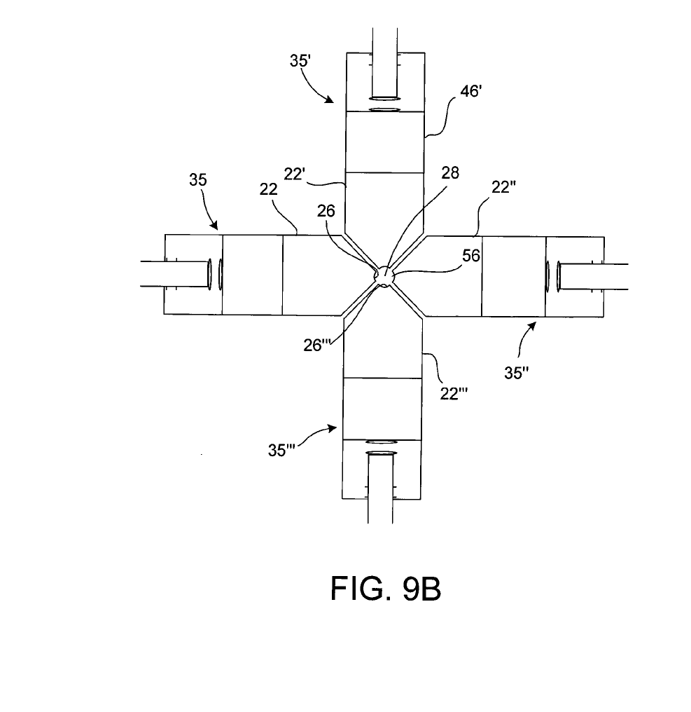

Once each blade device 22, 22′, 22″, . . . 22ⁿ has independently displaced from the respective crimp position to the respective retracted position (i.e., FIGS. 8G and 9B), the drive assembly 30 is configured to displace the respective edge walls 26, 26′, 26″, . . . 26ⁿ, together as a unit, along respective predetermined second paths P₂ (e.g., FIGS. 10 and 11) from the respective retracted position (FIGS. 8G and 9B) back to the crimp position (FIGS. 8A and 9A). As will be described in greater detail below, it is this collective and simultaneous movement along the respective predetermined second paths P₂ that incrementally crimp (radially inward) at least a portion of the stent 21 from the first diameter toward the reduced second diameter.

This cycling of the blade devices 22, 22′, 22″, . . . 22ⁿ sequentially along the respective predetermined first path P₁ (e.g., FIGS. 10, 11) to the respective retracted position, and then simultaneously, together as a unit, along the respective predetermined second path P₂ back to the respective crimp position is repeated over and over. Accordingly, during each crimp cycle, the stent 21, which may or may not be disposed about a delivery device 31 (e.g., FIG. 8C), is incrementally crimped radially inwardly as it is simultaneously distally displaced incrementally along the common longitudinal 27 and drawn through the crimp aperture 28 from the respective proximal portions 23, 23′, 23″, . . . 23ⁿ to the respective distal portions 25, 25′, 25″, . . . 25ⁿ of the blade devices. Such incremental crimp cycling, as the blades simultaneously displace along their respective second paths P₂ as a unit significantly minimizes any shear forces imparted by the respective edge walls 26, 26′, 26″, . . . 26ⁿ upon the corresponding outer surface of the stent. This is due in part to the movement of the blade devices 23, 23′, 23″, . . . 23ⁿ relative to the stent 21, which is essentially radially inward as opposed to any relative longitudinal displacement of the blade devices to the stent. This is particularly suitable for drug coated stents where it is desirable to minimize shear forces at the outer surface.

Referring back to FIGS. 1-3, the crimping assembly 20 is detailed having a housing assembly 32 that includes a plurality of housing plates 33, 33′, 33″, . . . 33ⁿ. In this example, each housing plate is similarly sized and mounted in a side-by-side relationship to one another, forming a polygonal shell-shaped housing assembly. Each plate also corresponds to an associated blade main assembly 35, 35′, 35″, . . . 35ⁿ that in turn corresponds to a respective blade device 22, 22′, 22″, . . . 22ⁿ. It will be appreciated, of course, that such one-to-one correspondence is not required and need not be the case.

The housing plates of the polygonal-shaped housing assembly are configured to define an interior space 36, and further adapted to support each blade main assembly 35 in a manner positioning the respective blade devices 22, 22′, 22″, . . . 22ⁿ within the interior space 36. Moreover, the housing plates 33, 33′, 33″, . . . 33ⁿ facilitate orientation of the devices blade in opposed cooperative relationship to one another, radially about the common longitudinal axis 27, to collectively define the crimp aperture 28.

Referring now to FIGS. 1-6, the blade main assemblies 35, 35′, 35″, . . . 35ⁿ will be described in detail. Briefly, while at least two blade main assemblies need to be employed, the Figures shown and described illustrate four blade main assemblies 35, 35′, 35″, 35′″, each oriented about 90° apart relative to one another. It will be appreciated, however, that more blade main assemblies may be employed, within the space limitations, increasing the crimping accuracy and continuity of the crimp. However, the number of parts also significantly increases along with design complexity.

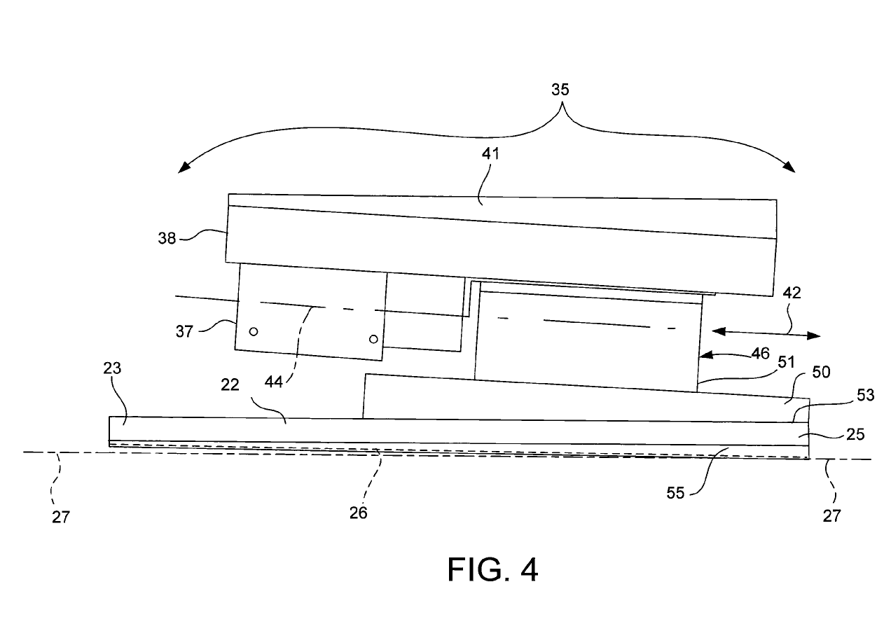

Due to the identical or substantially identical nature of each opposed blade main assembly, only one blade main assembly 35 of the four blade main assemblies 35, 35′, 35″, 35′″ (shown in the Figures) will be detailed for reasons of clarity. Hence, as best illustrated in FIGS. 4-6, the blade main assembly 35 includes a respective drive device 37 of the drive assembly 30 that drives the relative displacement between the respective blade device 22 and the housing assembly 32. The blade main assembly 35 includes an elongated support table 38 configured to support the respective drive device 37 thereon. An elongated rail 40 upstands from the table 38, facing toward the respective blade device 22, and extends generally in a direction longitudinally along the support table. To mount the support table 38 to the respective housing plate 33, a housing spacer 41 is provided that spaces the support table interiorly away from an interior wall of the housing plate. This spacer 41 also orients the drive device, and hence, the blade device 22 relative to the remaining opposed blade devices and the crimp aperture 28. For example, as best shown in FIG. 4, the housing spacer 41 is wedge shaped, slanting the distal portion of the support table radially inward toward the common longitudinal axis 27 of the crimp aperture 28.

In one specific embodiment, the drive device 37 is provided by a voice coil or solenoid device capable of selective reciprocal substantially linear displacement along a respective longitudinal axis thereof (i.e., in a direction of arrow 42 in FIG. 4).

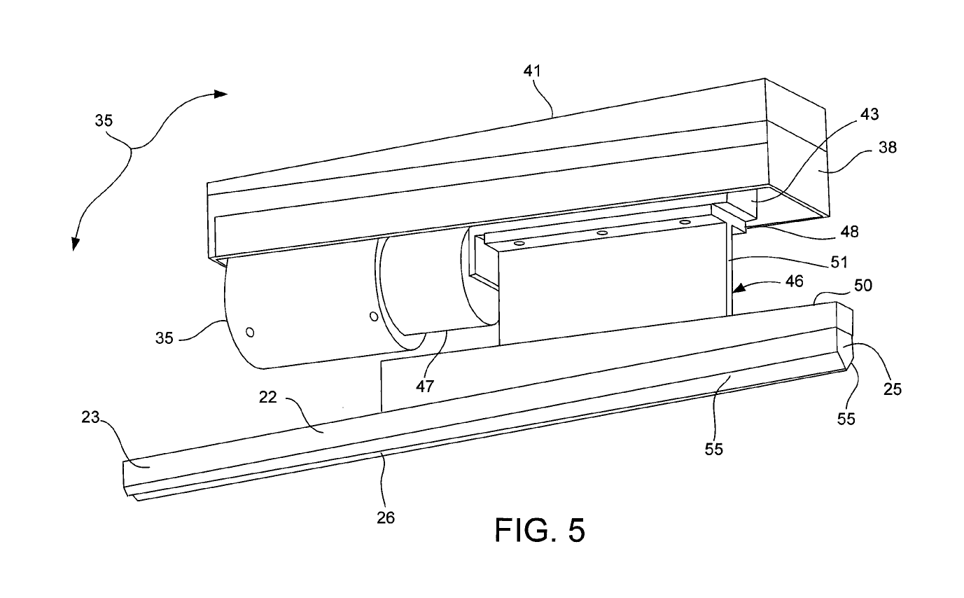

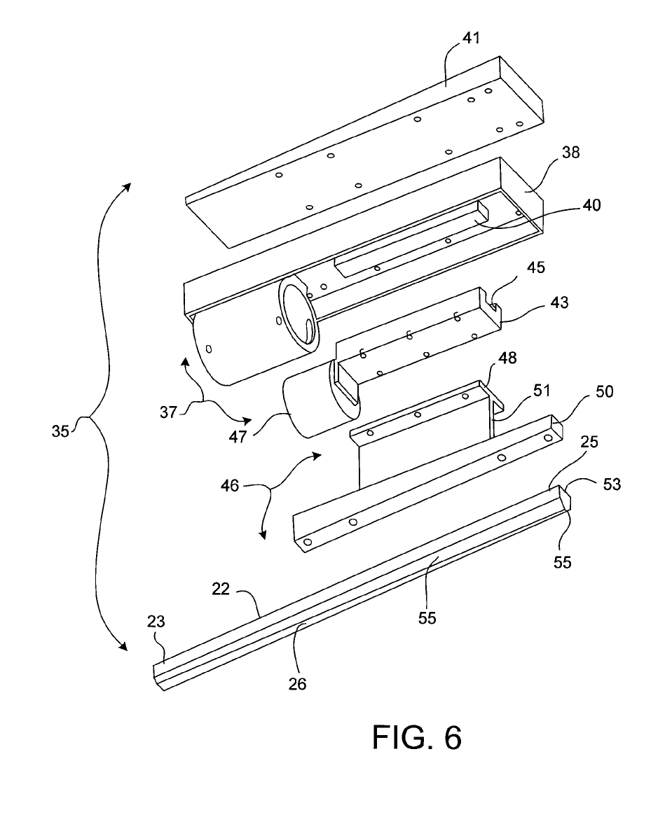

Applying a conventional linear tracking system, such as the elongated rail 40 upstanding from the support table 38 and a corresponding slider carriage 43 (FIGS. 5 and 6) cooperating with the elongated rail for sliding reciprocal movement in the direction of arrow 42 in FIG. 4. Briefly, the slider carriage includes an elongated slot 45 formed for sliding receipt of the elongated rail 40 therein for aligned substantially linear movement. One portion of the slider carriage 43 is mounted to a reciprocating shaft 47 (along shaft axis 44) of the drive device 37, while another portion of the slider carriage is coupled to the corresponding blade device 22.

A blade spacer 46 may be provided between the blade device 22 and the slider carriage 43 that is deployed to orient the blade device 22 relative to the remaining opposed blade devices 22′, 22″, 22′″ and the common longitudinal axis 27 of crimp aperture 28. Each blade spacer 46 includes a carriage mount flange 48; a blade mount flange 50 and an extension arm 51 extending between the carriage mount flange and the blade mount flange 50. FIGS. 4-6 best illustrates that the blade mount flange 50, for example, is wedge-shaped or tapered in a manner orienting the movement of the respective blade device 22, relative to the remaining opposed blade devices 22′, 22″, 22′″. Accordingly, the corresponding housing spacer 41 of the blade main assembly 35 facilitates the orientation of the drive device 37, and hence the reciprocating movement (along the direction of arrow 42 in FIGURE YY) of the slider carriage 43, via shaft 47, relative to the housing assembly 32 and remaining opposing blade main assemblies 35′, 35″, 35′″, while the corresponding blade spacer 46 orients the blade device 22 relative to the opposed remaining blade devices 22′, 22″, 22′″ and common longitudinal axis 27 of the crimp aperture 28.

Accordingly, as will be described in the collective operation of this specific embodiment below, due to the substantially linear reciprocal movement of the single drive device 37, the respective predetermined first path P₁ (e.g., FIG. 10) of the respective blade device 22, from the respective crimp position to the respective retracted position, and the respective predetermined second path P₂ (e.g., FIG. 10), from the respective retracted position back to the respective crimp position are coincident, albeit in opposite directions. Moreover, the direction of the first path and the second path are determined by the orientation of the respective blade device relative to the corresponding drive device (i.e., along the direction of arrow 42 in this configuration.)

It will be appreciated, however, that while the movement of the respective blade device 22 along the predetermined first path and the predetermined second path between the crimp position and the retracted position are both substantially linear and coincident, more sophisticated non-linear paths may be implemented, especially with respect to the respective predetermined first paths (e.g., P₁. in FIG. 11). As will be described in greater detail below, such non-linear motion is applied to reduce or minimize the shear stress between the blade and the loaded stent disposed in the crimp aperture 28.

Referring now to FIGS. 5 and 6, the blade device 22 is shown and described in detail. Each blade device 22 is elongated and extends from a proximal portion 23 to a distal portion 25, and includes a backside wall 53 configured to mount to the blade mounting flange 50 of the blade spacer 46. The blade device 22 further includes a pair of opposing side walls 55, each converging inwardly toward one another, and terminating at the distal contacting edge wall 26 that is formed and dimensioned to contact and incrementally crimp at least a portion of a loaded stent 21. The edge wall 26 extends generally in the direction of a longitudinal axis of the blade device 22 itself, albeit tapering radially inwardly (with respect to the collective crimp aperture 28 formed between the remaining opposing blade devices 22′, 22″, . . . 22ⁿ) from the proximal portion 23 to the distal portion 25 thereof.

To facilitate the formation of a substantially circular transverse cross-sectional dimension of the substantially cylindrical crimped stent 21 about the delivery device 31, the transverse cross-sectional dimension of the edge wall 26 is curvilinear, and preferably a circular sector. The arc length (radians) of the circular sector, of course, depends primarily upon the number of blade devices radially positioned about the common longitudinal axis 27 of the crimp aperture 28 defined. For example, at least two, and preferably at least four, main blade assemblies 35, 35′, 35″, 35′″ are employed. In this specific embodiment, hence, each edge wall 26 defines about a quarter circular sector each. When the four opposed blade devices 22, 22′, 22″, 22′″ are assembled in the crimp position (FIGS. 8A and 9A), the side walls 55 of the adjacent blades are directly opposed one another such that the tapered edge walls 26, 26′, 26″, 26′″ cooperate to form a substantially continuous conical frustum-shaped crimp aperture 28.

More than four blade assemblies may of course be implemented, increasing the crimping continuity and uniformity between the adjacent edges walls. It will be appreciated, however, that while the adjacent edge walls 26, 26′, 26″, 26′″ defining the crimp aperture, are preferably substantially continuous perimetrically, in the crimp position, small longitudinally extending gaps between the adjacent blade devices are permitted without departing from the true spirit and nature of the present invention. It will further be appreciated that the edge walls need not be conical frustum-shaped sectors. In fact, the transverse cross-sectional dimension of the edge walls could be relatively straight, as compared to curvilinear, in the transverse cross-sectional dimension, albeit tapering inwardly from the proximal portion to the distal portion. This would be especially true when a greater number blade devices are employed.

Each blade device may be constructed of a material or a combination of materials such as nylon, delrin, steel, aluminum, titanium, TEFLON®, plastics, composite materials, and other suitable materials. Such material selections depend in part upon the material properties, such as the thermo insulation, the thermo conductivity, and whether friction therebetween is low or high, etc. It is further contemplated that the blade device 22 may be constructed as a unitary member, or may be constructed of multiple pieces that may be assembled to form a unitary member. For example, the contacting edge walls employed to collectively crimp the stent may be provided by replaceable blade inserts or the like (not shown).

It is further contemplated that blade device 22 may include a coating disposed thereon. For example, blade device 22 may be coated with a material that reduces friction, increases hardness, or alters other mechanical properties of the device according to the present invention. To reduce friction between adjacent blades or to reduce friction between the edge walls and the stent to be crimped, by way of example, it is contemplated that the blade devices may be polished to a high degree in addition to or instead of coating the blade. For example, if it is desirable to form a blade of stainless steel, the blade may be constructed having a highly polished surface finish to reduce friction and to further reduce the possibility of scratching or otherwise damaging a stent to be crimped.

The translational movement of the blade devices 22, 22′, 22″, 22′″, as will be discussed, may be performed by a control unit (not shown). The control unit, for instance, may be provided by a computer or the like, wherein the computer includes a program designed to control the sequential and simultaneous movement of the plurality of blade devices along the predetermined first path P₁, from the crimp position to the retracted position, and along the predetermined second path P₂, simultaneously from the retracted position back to the crimp position.

Referring now to the sequence of FIGS. 8A-8G, 9A and 9B, the operation of one crimping cycle of the crimping assembly 20 will now be described in detail. Briefly, in accordance with the present invention, the crimping assembly shown and described herein may be utilized to reduce the diameter of a medical device (not shown) such as a stent from a first diameter to a reduced second diameter. In particular, the stent 21 may be comprised of either a balloon expandable or a self-expanding stents disposed around a delivery device 31, such as a balloon catheter, for example.

Initially, when the crimping assembly 20 is placed in the crimp position (FIG. 8A, 9A), the blade main assemblies 35, 35′, 35″, 35′″ orient and align the corresponding edge walls 26, 26′, 26″, 26′″ of the respective blade devices 22, 22′, 22″, 22′″ such that they collectively define and form a substantially continuous conical frustum-shaped crimp aperture 28. At a distal end of the aligned blade devices 22, 22′, 22″, 22′″ is an exit port 56 from the crimp aperture 28 to enable passage of the crimped stent from the crimp aperture as will be described. The exit port 56 from the crimp aperture is diametrically sized and dimensioned to impart the desired final crimp diameter upon the stent 21 and onto the delivery device 31 as they pass out of the crimping assembly.

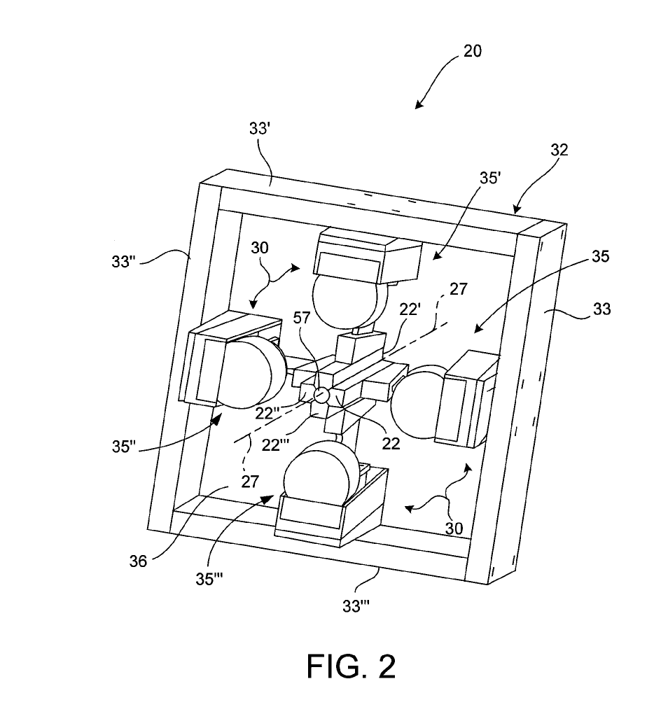

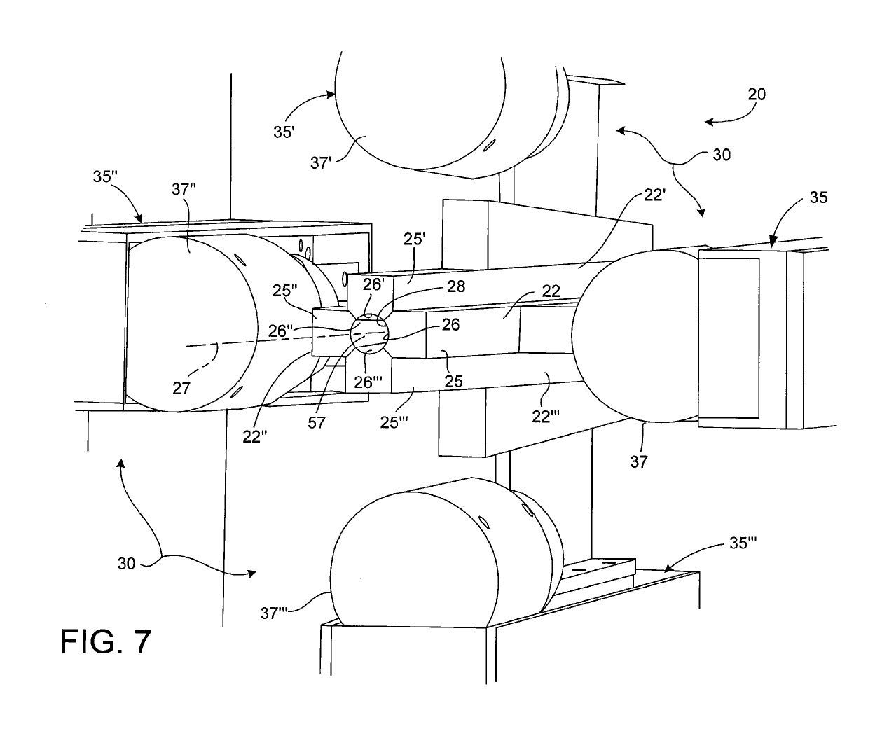

On an opposite proximal end of the aligned blade devices 22, 22′, 22″, 22′″ is an entrance port 57 into the crimp aperture 28 that enables sliding entrance of the uncrimped stent 21 therein for initial loading thereof (FIGS. 2 and 7). Thus, the diameter of the entrance port 57 into the conical frustum-shaped crimp aperture 28 is at least as large as that of the uncrimped first diameter of the stent. More preferably, the diameter of the entrance port 57 is greater than that of the uncrimped first diameter to permit receipt of at least a significant longitudinal length of the stent into at least a proximal portion of the crimp aperture 28. Accordingly since the stents themselves are resilient and self-expanding, they can be slightly radially compressed to permit initial placement and positioning in the crimping assembly without damage to the stent or coating. Moreover, some frictional resistance against the collective edge walls 26, 26′, 26″, 26′″, when all the respective blade devices are in the retracted position (FIGS. 8F 8G and 9B), is permissible and even necessary, to enable the collective blade devices to move the stent/delivery device along the second path P₂ (e.g., FIGS. 10 and 11) as a unit.

As mentioned, the distal end uncrimped stent 21, in the first diameter about the delivery device 31, is initially positioned or slid through the entrance port 57 of the crimp aperture at a proximal end of the placed. Preferably, the width of the inwardly tapered crimp aperture 28, at the proximal portion thereof, is sufficiently sized to receive a significant length, if not all, the stent therein for stability of the stent and delivery device, during the crimping process. According, the stent 21/delivery device 31 combinations should be manually inserted into the crimp aperture 28, via the entrance port 57 until a small degree of resistance is felt. Too much resistance may indicate crimping damage to the stent at the distal end thereof, via longitudinal sliding contact with the edge walls. Such shear stress along the outer surface of some stents may be problematic; such as for example drug coated stents.

Once the stent/delivery device combination is initially place in the crimp aperture through the entrance port 57, the crimping cycle can commence. Referring now to FIG. 8B, the first main blade assembly 35 is actuated, via the respective drive device 37, retracting the respective blade device 22 along the first path P₁ (e.g., FIGS. 10, 11) from the respective crimp position (FIG. 8A) to the retracted position (FIG. 8B). It will be appreciated that in the most simplistic form, the first path P₁ and the second path P₂ between the respective crimp position and the retracted position, as shown by the arrow in one direction for P₁ and by the arrow in the opposite direction for P₂ in FIG. 10, is a substantially linear. In this manner, only a single drive device 37 may be required, having a selective reciprocating linear motion, such as the voice coil or solenoid device shown in the FIGURES.

The first path P₁ the second path P₂ are also substantially contained within the respective plane that longitudinally bisects the respective blade device, and that extends through the common longitudinal axis 27 (FIGS. 10 and 11). By way of example, referring to FIG. 10, point PT_{C} represents a random point along the edge wall 26 of blade device 22, in the crimped position, while PT_{R} represents that same point along the edgewall 26 as it has moved along the first path P₁ to the retracted position. Hence, as mentioned and as illustrated, this predetermined first path P₁ is both substantially linear, and substantially contained within the bisecting plane.

Moreover, at a minimum, the slope of movement of the blade device, relative to the common longitudinal axis 27, along the first path P₁ from the point PT at the crimp position (PT_{C}) to and the retracted position (PT_{R}), is at least substantially equal to the slope of the taper of the respective edge wall 26. Any such slope less than that of the edge wall my induce too much shear stress on the outer surface of the stent as the edge wall 26 slides thereagainst during movement from the respective crimp position and the respective retracted position along the substantially linear first path P₁.

In fact, in accordance with one specific embodiment of the present invention, the slope of this linear first path P₁, relative to the common longitudinal axis 27, is actually greater than that of the taper of the respective edge wall 26. Hence, as represented in FIG. 10, during movement of the respective blade device along the substantially linear first path P₁, the representative PT along the respective edge wall 26 (at the crimp position (PT_{C})) actually pulls away (radially) from outer surface of the stent 21 (not shown) positioned in the crimp aperture 28 (at the retracted position (PT_{R})). Accordingly, the slope of the first path P₁ is slightly steeper than that of the taper of the corresponding edge wall 26.

Briefly, it will further be appreciated that the length of the first path P₁, as well as that of the second path P₂, shown in the drawings, are exaggerated for the reason of illustration. While the actual length of the first path P₁ may vary, such length may generally be in the range mm.

As indicated above and as illustrated in this configuration, the respective drive devices 37, 37′, 37″, . . . 37ⁿ of the drive assembly 30 provide a substantially linear first path P₁ and second path P₂ similar to that shown in FIG. 10. In accordance with the present invention, however, a more sophisticated two dimensional displacement may be implemented to more positively move the blade device 22 radially way from contact with the outer surface of the stent during retraction (along the first path P₁ of for example the point PT) from the respective crimp position to the respective retracted position. In this manner, any surface shear impinged upon the outer surface of the stent is further minimizing, if not eliminated.

By way of example, as shown in FIG. 11, the first path P₁ may include components in two dimensions (i.e., a vertical and a horizontal component) between the respective crimp position and the retracted position, as long as both components are contained in the respective plane extending through the common longitudinal axis 27 of the crimp aperture. The two dimensional first path P₁ of this embodiment initially includes a vertical component lifting the edge wall 26 of the respective blade device 22 out of contact with, or significantly reduced contact with, the outer surface of the stent 21. Such vertical movement is represented by the first leg L₁ of the first path P₁ extending generally in a direction radially away from the common longitudinal axis 27.

The next component, represented by the second leg L₂ of the first path P₁ that extends generally parallel to the common longitudinal axis 27 in a proximal direction. As shown in FIG. 11, this horizontal component is applied to generally determine the axial distance between the respective crimp position and the retracted position of the first path P₁.

The last leg L₃ of the first path P₁ represents another vertical component, moving the edge wall 26 radially downward in a direction toward the common longitudinal axis and into contact with the outer surface of the stent 21, to the final retracted position of this blade device 22. Contact with the outer surface of the stent during this last leg L₃ is also assured since these stents are resilient and/or self-expanding. As will be described, this contact is necessary to enable movement of the stent/delivery assembly along the second path P₂.

It will be appreciated that any such radial displacement along the first path P₁ (in all first path embodiments) depends upon the total number of blades applied. That is, the greater the number of blades, the smaller the radial expansion of the stent when the contained in the crimp aperture. Thus, the greater the number of blades, the smaller the radial displacement, as well as a smaller axial movement. This is beneficial when the process is near completion at the exit of the stent/delivery device from the crimp aperture. At this portion of the crimp procedure, smaller axial (and radial) increments are advantageous since the crimp of the stent is already at an advanced stage. Accordingly, it follows that near the beginning of the crimp process, the increments of the first path P₁ and corresponding second path P₂ can be larger than the increments near the end of the crimp process.

To execute such two dimensional movement, at least two cooperating drive devices (not shown) may be required for each main blade assembly, each enabling reciprocal movement in a substantially linear direction, albeit directed 90° apart for example. Through cooperative movement, even more complicated arc, elliptical, or circular first path P₁ mappings can be generated.

In accordance with the present invention, the remaining blade devices 22′, 22″, 22′″ are sequentially operated in a similar manner with no particular order. By way of example, FIG. 8D illustrates the blade device 22′ moved similarly along the respective first path P₁ to the respective retracted position, while FIG. 8E represents the same for the third blade device 22″. Finally, FIG. 8F illustrates the last blade device 22′″ moved along the first path to the respective retracted position. It will be appreciated that while the individual blade devices are sequentially moved proximally to the respective retracted position, the stent/delivery device disposed in the crimp aperture 28 will maintain its axial position relative to the housing assembly. In other words, the friction between the outer surface of the stent and the edge walls of the remaining blade devices, not currently participating in the movement to the retracted position, will cooperate to retain stent axially along the crimp aperture 28. In fact, as partially illustrated, expanding stents such as a Nitnol self-expanding stent 21 will radially expand a limited amount to assure the requisite frictional contact without detriment to the outer surface of the stent, or its coating. Hence, the crimper design of the present invention will provide the minimum damage during contact between the blades and the coating surfaces of the stent. This is especially true with the conical shape of crimp aperture due to the tapered edge of blades versus a case where the crimp aperture is substantially cylindrical, and the blades are relatively flat or substantially parallel to the common axis.

Once all the blade devices 22, 22′, 22″, 22′″ are oriented in the retracted position (FIGS. 8G, 9B), the blade devices are moved along the second path P₂ from the respective retracted position back to the crimp position, simultaneously and together as a unit (FIGS. 8A and 9A). It is this movement of the collective blade devices (both axially forward and radially inward) along the respective second paths P₂ that generates the radial inward crimping force urged upon the outer surface of the stent. The outer surface of the stent, however, only experiences the inward crimping force since, as the blade devices move axially along the respective second paths from the respective retracted position to the respective crimp position, the friction between the stent outer surface and the edge walls cause the stent/delivery device to be dragged axially with the collective blade devices. Accordingly, there is no lateral shear stress between the edge walls and the stent outer surface.

As this crimping cycle is repeated over and over, the stent/delivery device combination is incrementally moved axially along the crimp aperture 28 from the proximal portion 23 to the distal portion 25. As the stent is moved axially along the crimp aperture, the edge walls incrementally crimp the stent from the first diameter to the reduced second diameter. Hence, to uniformly crimp along the entire length of the stent/delivery device, due to the tapered nature of the collective edge walls, it may be necessary to pass the stent fully through the crimp aperture 28 and out of the exit port 56. The number of crimp cycles necessary to perform this procedure will of course depend upon the length of the stent and the longitudinal length of each cycle.

In one specific embodiment, when perhaps 6 or more blades are applied, the movement of the blades needs to be independent, and not entirely sequential. For example, if the crimping assembly employed 8 blade assemblies, every other blades (e.g., 1, 3, 5 and 7) could move relatively simultaneously along the first path P₁ from the respective crimp position to the respective retracted position. Subsequently, blades 2, 4, 6 and 8 could move relatively simultaneously along the first path P₁. The key in this situations is that with a sufficient number of remaining blades circumferentially surrounding the loaded stent, to axially as well as radially retain is in place, the first set of blades are retracted simultaneously.

In another specific embodiment of the present invention, it is further contemplated that the crimping assembly may additionally include a chiller unit (not shown) wherein the chiller unit is configured to chill or cool the crimp aperture 28 collectively formed between the blade devices 22, 22′, 22″, . . . 22ⁿ. This is advantageous when stents that must be cooled or chilled in order to reduce their diameters. For example, Nitinol stents must be cooled in order to reduce the diameter of the stent from an expanded diameter to a delivery diameter. The chiller may be integrally formed with the crimping assembly or may be a separate component that may be designed to work in conjunction with the apparatus.

Further still, it is contemplated that the blade devices may be modified in order to function correctly with the chiller unit. The crimp aperture 28 itself, formed by edge walls 26, 26′, 26″, . . . 26ⁿ, is a highly insulated chamber, and is suitable for cryogenic or other thermotreating processing. By providing an end cap or the like, the exit port 56 of the crimp aperture 28 can be sufficiently sealed. A hub portion of the end cap may be sized for a friction fit into the exit port, and an O-ring may further be provided to form a fluid tight seal. A set of access ports may extend through the end cap that provide access to the crimp aperture 28 for selective cooling thereof.

Another embodiment includes cooling of the blade devices themselves through cooling channels or passages. In this configuration, the blade devices could include communication orifices or the like that communicate a coolant from the coolant channels with the crimp aperture for cooling thereof.

Although the present invention has been described in connection with the preferred form of practicing it and modifications thereto, those of ordinary skill in the art will understand that many other modifications can be made thereto within the scope of the claims that follow. Accordingly, it is not intended that the scope of the invention in any way be limited by the above description, but instead be determined entirely by reference to the claims that follow.

What is claimed is:

1. A stent crimping assembly for crimping a stent from a first diameter to a reduced second diameter, said crimping assembly comprising:
a set of two or more blade devices each having a proximal portion and a downstream distal portion, and a respective edge wall extending from the proximal portion to the distal portion, each respective edge wall being oriented relative to one another, in a respective crimp position, to collectively define an elongated conical-shaped crimp aperture, each edge wall tapering inwardly along a common longitudinal axis from the respective blade proximal portion toward the distal portion, the conical-shaped crimp aperture at the respective proximal portion of each blade being formed for receipt of at least a portion of the stent in the first diameter;
a drive assembly associated with each blade device and configured to independently displace each blade edge wall in a sequential manner substantially along a respective predetermined first path from the respective crimp position to a respective retracted position, oriented a predetermined incremental distance from said crimp position in a respective proximal direction.

2. The stent crimping assembly according to claim 1, wherein
a transverse cross-sectional dimension of each respective edge wall is circular sector-shaped such that the collective transverse cross-sectional dimension of the opposed two or more blades, in the crimp position, is substantially circular shaped.

3. The stent crimping assembly according to claim 2, wherein
each respective edge walls, in the crimp position, is configured to cooperate with an adjacent edge wall to form a substantially continuous circular surface to define a substantially continuous crimp aperture.

4. The stent crimping assembly according to claim 1, wherein
the set of blade devices includes four blade devices each off-set substantially about 90° about the common longitudinal axis from an adjacent blade device.

5. The stent crimping assembly according to claim 1, wherein
after each blade device has sequentially displaced from the respective crimp position to the respective retracted position, the drive assembly is configured to displace the respective edge walls along respective predetermined second paths, as a unit, from the respective retracted position back to the crimp position, displacing the stent substantially along the common longitudinal axis, relative to the crimping assembly, in a direction toward the respective distal portions.

6. The stent crimping assembly according to claim 5, wherein
the respective predetermined second path each of each blade edge wall from the respective retracted position back to the crimp position is substantially linear.

7. The stent crimping assembly according to claim 6, wherein
each respective predetermined second path is tapered radially inwardly toward the common longitudinal axis such that the portions of the stent are crimped from the first diameter to the reduced second diameter.

8. The stent crimping assembly according to claim 7, wherein
each respective predetermined second path is substantially parallel to a longitudinal axis of the respective edge wall.

9. The stent crimping assembly according to claim 5, wherein
each respective first and predetermined second path of each respective edge wall is contained in a respective plane extending through the common longitudinal axis.

10. The stent crimping assembly according to claim 1, wherein
the respective predetermined first path each of each blade edge wall from the respective crimp position to the retracted position is substantially linear.

11. The stent crimping assembly according to claim 10, wherein
each respective predetermined first path is tapered radially outwardly away the common longitudinal axis.

12. The stent crimping assembly according to claim 10, wherein
each respective predetermined first path is substantially parallel to the common longitudinal axis of the crimp aperture.

13. The stent crimping assembly according to claim 1, wherein
each respective predetermined first path of each respective edge wall is contained in a respective plane extending through the common longitudinal axis.

14. The stent crimping assembly according to claim 1, wherein
the respective predetermined first path each of each blade edge wall from the respective crimp position to the retracted position is substantially non-linear, initially extending in a direction radially away from the common longitudinal axis.

15. The stent crimping assembly according to claim 1, wherein
each respective edge wall from the respective blade first portion to the respective blade distal portion is gradually sloped, relative to the common longitudinal axis.

16. The stent crimping assembly according to claim 1, wherein
said drive assembly includes a respective drive device independently associated with corresponding blade device, and a control system controlling operation and sequence of each drive device.

17. A stent crimping assembly for crimping a stent from a first diameter to a reduced second diameter, said crimping assembly comprising:
a set of two or more blade devices each having a first end and a second end, and a respective edge wall in opposed relationship to one another, each respective edge wall tapering inwardly from the respective one ends to the respective second ends, relative to a common longitudinal axis of an elongated crimp aperture collectively defined by the opposed edge walls in a respective crimp position, each said respective one ends collectively defining a receiving port into said crimp aperture formed for receipt of at least a portion of the stent in the first diameter;
a respective drive assembly associated with each blade device and configured to sequentially displace each blade substantially along a respective predetermined path from the respective crimp position to a respective retracted position, oriented a predetermined incremental amount substantially along the common longitudinal axis of the crimp aperture and in a relative direction toward the respective one ends, sequentially diametrically reducing at least a portion of the stent from the first diameter to the reduced second diameter.

18. The stent crimping assembly according to claim 17, wherein
a transverse cross-sectional dimension of each respective edge wall is circular sector shaped such that the collective transverse cross-sectional dimension of the opposed two or more blades, in the crimp position, is substantially circular shaped.

19. The stent crimping assembly according to claim 18, wherein
each respective edge walls, in the crimp position, is configured to cooperate with an adjacent edge wall to form a substantially continuous circular surface to define a substantially continuous crimp aperture.

20. The stent crimping assembly according to claim 17, wherein
the set of blades includes four blades in opposed relationship to one another.

21. The stent crimping assembly according to claim 17, wherein after each blade device has sequentially displaced from the respective crimp position to the respective retracted position, each respective drive assembly is configured displace the respective blade devices substantially along the common longitudinal axis, as a unit, from the respective retracted position back to the crimp position, displacing the stent, relative to the crimping assembly, in the direction toward the respective second ends.

22. The stent crimping assembly according to claim 17, wherein
each respective drive assembly is configured to displace each respective blade device, from said respective crimp position to the respective retracted position, along a substantially linear predetermined path substantially along the common longitudinal axis.

23. The stent crimping assembly according to claim 17, wherein
each respective drive assembly is configured to initially displace the respective blade device, from said respective crimp position to the respective retracted position, along the predetermined path in a direction radially away from the common longitudinal axis.
